(19) United States
(12) Patent Application Publication
(10) Pub. No.: US 2024/0296910 A1
(43) Pub. Date: Sep. 5, 2024

(54) SYSTEMS AND METHODS FOR IDENTIFICATION OF ION CHANNELS

(71) Applicant: Ohio State Innovation Foundation, Columbus, OH (US)

(72) Inventors: Mustafa Demirtas, Bursa (TR); Przemyslaw Radwanski, Columbus, OH (US)

(21) Appl. No.: 18/442,501

(22) Filed: Feb. 15, 2024

Related U.S. Application Data
(60) Provisional application No. 63/445,866, filed on Feb. 15, 2023.

(51) Int. Cl. G16B 40/20; G01Q 60/44

(52) U.S. Cl. CPC G16B 40/20; G01Q 60/44

(57) ABSTRACT

Systems and methods for identification of ion channels are described herein. In some implementations, the techniques described herein relate to a computer-implemented method including: receiving a single channel activity signal associated with an ion channel of a cell; performing a time-domain analysis on the single channel activity signal; and identifying, based on the time-domain analysis, an isoform of the ion channel.

### CROSS-REFERENCE TO RELATED APPLICATIONS

This application claims the benefit of U.S. provisional patent application No. 63/445,866, filed on Feb. 15, 2023, and titled “SYSTEMS AND METHODS FOR IDENTIFICATION OF ION CHANNELS,” the disclosure of which is expressly incorporated herein by reference in its entirety.

### STATEMENT REGARDING FEDERALLY FUNDED RESEARCH

This invention was made with government support under Grant no. NS121234 and HL155378 awarded by the National Institutes of Health. The government has certain rights in the invention.

### BACKGROUND

Ions play an important role in nerve and muscle excitability. Excitation, or an electrical signal, is created by the transport of ions through ion channels, which are the primary excitatory elements of the membranes of nerves, muscles, and other tissue cells. Electrically excitable cells maintain high intracellular K⁺ and low Na⁺ concentrations compared to extracellular concentrations. A concentration gradient leads to an electric potential. The main difference between electrically excitable cells and others is that the ion channels of the first are sensitive to the potential difference between the internal and external surfaces of their membranes. The response of the channels to changes in the membrane potential occurs within a few milliseconds through a regenerative increase in the permeability to Nat. These channels are composed mainly of proteins that form macromolecular pores around 0.3-0.8 nm in diameter that can open and close synchronously, causing electrical signals resulting in neural responses as well as muscle activation. Open highly selective ion channels allow certain types of ions to enter along an electrochemical gradient with a flow of approximately 105 ions/s.

Today, these channels are considered one of the four most important protein families in drug discovery. There is still great uncertainty and much remains to be discovered in this important protein family. In particular, voltage-gated sodium channels are critical elements in action potential initiation and propagation in excitable cells, as they are responsible for the initial membrane depolarization. These channels consist of a highly processed a-subunit associated with auxiliary B-subunits. The pore-forming subunit is sufficient for functional expression, but the B-subunits shape the kinetics and voltage dependence of channel gating. Several different types of sodium channels have been identified by electrophysiological recording, biochemical purification, and molecular cloning.

Current methods for identifying isoforms of existing ion channels often prove inadequate, highlighting a crucial need for more advanced methodologies. The limitations of conventional approaches hinder our ability to accurately discern the diverse isoforms, impeding progress in understanding the intricate workings of voltage-gated ion channels [1-2]. Furthermore, the complexity of ion channel composition within the physiological milieu makes it extremely difficult to identify the ion channels and their isoforms that may be targeted by lead pharmacological compounds. Recognizing these challenges, a consensus within the scientific community is growing, suggesting that traditional identification techniques lack the precision required for comprehensive analysis.

It is therefore desirable to provide signal processing-based methods to analyze single channel activity signals to identify the isoforms of ion channels. Such methods may improve the identification and screening of the most essential protein families for industrial pharmaceutical drug research, for example.

### SUMMARY

Systems and methods for identification of ion channels are described herein. In some implementations, the techniques described herein relate to a computer-implemented method including: receiving a single channel activity signal associated with an ion channel of a cell; performing a time-domain analysis on the single channel activity signal; and identifying, based on the time-domain analysis, an isoform of the ion channel.

In some implementations, the time-domain analysis includes dynamic time warping (DTW). Optionally, the time-domain analysis includes calculating a respective Euclidean distance for one or more fluctuations of an amino-acid sequence using DTW.

In some implementations, the isoform of the ion channel is one of a sodium channel (Nav), a potassium channel (Kv), a calcium channel (Cav), or a chloride channel (ClC).

In some implementations, the single channel activity signal is measured using a cell-attached patch-clamp system or an ion conductance microscopy-guided smart patch-clamp system.

In some implementations, the step of performing the time-domain analysis on the single channel activity signal further includes extracting at least one time-domain feature, and the method further includes: inputting, into a trained machine learning model, the at least one time-domain feature; and predicting, using the trained machine learning model, the isoform of the ion channel.

In some implementations, the method further includes comparing the isoform of the ion channel identified based on the time-domain analysis to the isoform of the ion channel predicted by the trained machine learning model.

In some implementations, the at least one time-domain feature comprises a Euclidean distance for one or more fluctuations of an amino-acid sequence.

In some implementations, the trained machine learning model is a supervised learning model. For example, the supervised learning model is a decision tree classifier, a support vector machine (SVM), a k-nearest neighbors (KNN) classifier, a Naïve Bayes' classifier, or an artificial neural network.

In some implementations, the techniques described herein relate to a computer-implemented method including: receiving a single channel activity signal associated with an ion channel of a cell; performing a frequency-domain analysis on the single channel activity signal; and identifying, based on the frequency-domain analysis, an isoform of the ion channel.

In some implementations, the frequency-domain analysis includes fast Fourier transform (FFT) or discrete Fourier transform (DFT). Optionally, the frequency-domain analysis includes a determining a power spectrum of the single channel activity signal using FFT or DFT.

In some implementations, the isoform of the ion channel is one of a sodium channel (Nav), a potassium channel (Kv), a calcium channel (Cav), or a chloride channel (ClC).

In some implementations, the single channel activity signal is measured using a cell-attached patch-clamp system or an ion conductance microscopy-guided smart patch-clamp system.

In some implementations, the step of performing the frequency-domain analysis on the single channel activity signal further includes extracting at least one frequency-domain feature, and the method further includes: inputting, into a trained machine learning model, the at least one frequency-domain feature; and predicting, using the trained machine learning model, the isoform of the ion channel.

In some implementations, the method further includes comparing the isoform of the ion channel identified based on the frequency-domain analysis to the isoform of the ion channel predicted by the trained machine learning model.

In some implementations, the at least one frequency-domain feature comprise a power spectrum.

In some implementations, the techniques described herein relate to a computer-implemented method, wherein the trained machine learning model is a supervised learning model. For example, the supervised learning model is a decision tree classifier, a support vector machine (SVM), a k-nearest neighbors (KNN) classifier, a Naïve Bayes' classifier, or an artificial neural network.

In some implementations, the techniques described herein relate to a computer-implemented method including: receiving a single channel activity signal associated with an ion channel of a cell; inputting one or more features associated with the single channel activity signal into a trained machine learning model; and predicting, using the trained machine learning model, an identity of an isoform of the ion channel.

In some implementations, the method further includes performing a time-domain analysis on the single channel activity signal to extract at least one time-domain feature, where the one or more features input into the trained machine learning model include the at least one time-domain feature.

In some implementations, the time-domain analysis includes dynamic time warping (DTW).

In some implementations, the method, further includes performing a frequency-domain analysis on the single channel activity signal to extract at least one frequency-domain feature, where the one or more features input into the trained machine learning model include the at least one frequency-domain feature.

In some implementations, the frequency-domain analysis includes fast Fourier transform (FFT) or discrete Fourier transform (DFT).

In some implementations, the one or more features input into the trained machine learning model include raw data.

In some implementations, the trained machine learning model is a supervised learning model. Optionally, the supervised learning model is a decision tree classifier, a support vector machine (SVM), a k-nearest neighbors (KNN) classifier, a Naïve Bayes' classifier, or an artificial neural network.

In some implementations, the isoform of the ion channel is one of a sodium channel (Nav), a potassium channel (Kv), a calcium channel (Cav), or a chloride channel (ClC).

In some implementations, the single channel activity signal is measured using a cell-attached patch-clamp system or an ion conductance microscopy-guided smart patch-clamp system.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flow chart illustrating example operations for a time-domain-based method for identifying ion channel isoforms according to an implementation described herein.

FIG. 2 is a flow chart illustrating example operations for a frequency-domain-based method for identifying ion channel isoforms according to an implementation described herein.

FIG. 3 is a flow chart illustrating example operations for a machine learning-based method for identifying ion channel isoforms according to an implementation described herein.

FIG. 4 is an example computing device.

FIGS. 5A-5F illustrate KS test statistic between all CDFs for both FFT and DTW-based methods according to the examples described herein. FIG. 5A illustrates all KS statistics using DTW-based method. FIG. 5B illustrates KS statistic for CHO expressing Nav1.6 (sometimes referred to herein as “CHO1.6”) fraction prediction using DTW-based method. FIG. 5C illustrates KS statistic for CHO expressing Nav1.5 (sometimes referred to herein as “CHO1.5”) fraction prediction using DTW-based method. FIG. 5D illustrates all KS statistics using FFT-based method. FIG. 5E illustrates KS statistic for CHO1.6 fraction prediction using FFT-based method. FIG. 5F illustrates KS statistic for CHO1.5 fraction prediction using FFT-based method.

FIGS. 6A-6F show the calculated FFT results for different channels and genotypes according to the examples described herein. FIGS. 6A-6D illustrate results for CHO1.6, CHO1.5, cardiac-specific Naᵥ1.6 knockout, and WT, respectively.

FIG. 7 is Table 1, which illustrates predicted fraction of Nav1.6 channels for known mixed datasets according to an example described herein.

FIG. 8 is Table 2, which illustrates predicted fraction of Nav1.5 channels for known mixed datasets according to an example described herein.

FIG. 9 is Table 3, which illustrates overall results of both Nav1.5 and Nav1.6 fraction prediction for the mixed datasets using the DTW-based method according to an example described herein.

FIG. 10 is Table 4, which illustrates overall results of both Nav1.5 and Nav1.6 fraction prediction for the mixed datasets using FFT based method according to an example described herein.

FIG. 11 is Table 5, which illustrates overall results of both Nav1.5 and Nav1.6 fraction prediction for the mixed datasets using DTW and FFT-based methods according to an example described herein.

FIG. 12 is Table 6, which illustrates machine learning predictions using the features extracted using FFT according to an example described herein.

FIG. 13 is Table 7, which illustrates machine learning predictions using the features extracted using DTW according to an example described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for identification of sodium channels, it will become evident to those skilled in the art that the implementations are not limited thereto, but are applicable for identification of other ion channels including other voltage-gated ion channels, ligand-gated ion channels such as calcium-activated potassium channels, acetylcholine-gated ion channels such as acetylcholine-gated potassium channels, hyperpolarization-activated, nucleotide-gated channels (HCN), etc.

As used herein, the terms “about” or “approximately” when referring to a measurable value such as an amount, a percentage, and the like, is meant to encompass variations of +20%, +10%, +5%, or +1% from the measurable value.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes' classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

As described above, ion channels (e.g., sodium channels) are critical elements in action potential initiation and propagation in excitable cells, as they are responsible for the initial membrane depolarization. These channels consist of a highly processed a-subunit associated with auxiliary B-subunits. The pore-forming subunit is sufficient for functional expression, but the B-subunits shape the kinetics and voltage dependence of channel gating. For example, the nine mammalian sodium channel forms that have been identified and functionally expressed share 5-15% amino acid sequence differences in the pore-forming domain, where the amino acid sequence is sufficiently different for identification using the systems and methods described herein. Patch-clamp technology now allows observation of nanoscale variation of open single-channel currents. Open channel current analysis can provide important information on how different conformational state transitions and biochemical modifications of ion channels, which may result from amino acid sequence differences, affect transport properties.

To bridge the gap of identifying ion channel isoforms by conventional approaches, there is a compelling demand for precisely identifying and characterizing ion channel isoforms. The systems and methods described herein use signal processing, which proves to be a powerful tool, to identify ion channel isoforms. For example, advanced filtering, machine learning, and statistical analysis among signal processing methods allow for deciphering the unique kinetic signatures associated with each isoform. To seamlessly integrate these tools into drug discovery processes, user-friendly software and validated computational models are imperative. As shown by the Examples, the systems and methods described herein have showcased immense potential in significantly enhancing the accuracy and efficiency of ion channel isoform identification.

As described herein, the systems and methods of this disclosure integrate signal processing-based techniques, presenting a more robust and sophisticated approach to unraveling the complexities of ion channel isoform identification, particularly within the realm of industrial pharmaceutical drug research. Through this integration, the systems and methods described herein can advance the methodologies employed in drug discovery, fostering a deeper understanding and more effective modulation of ion channels.

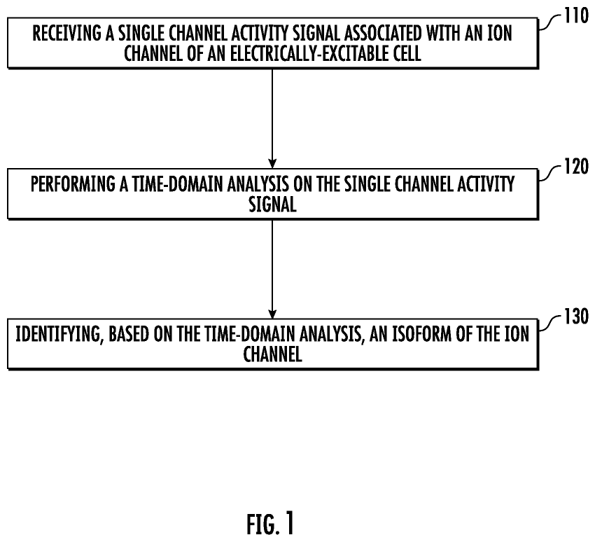

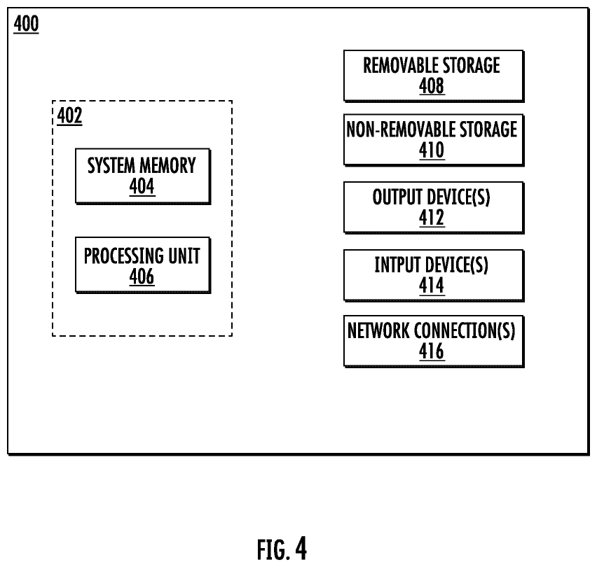

Referring now to FIG. 1, a flow chart illustrating example operations for a time-domain-based method for identifying ion channel isoforms is shown. This disclosure contemplates that the operations shown in FIG. 1 can be performed using a computing device (e.g., computing device 400 of FIG. 4). The time-domain-based signal processing approach of FIG. 1 provides improvements over conventional techniques for identifying ion channel isoforms. Such conventional techniques include genetic analysis (e.g. DNA sequencing), transcript analysis (e.g. RNA sequencing), protein analysis (e.g. Western Blotting, immunohistochemistry), and pharmacological means. Conventional techniques, however, have limitations and have proved to be inadequate as described above. In contrast, time-domain-based signal processing provides a solution that addresses the complexities of ion channel isoform identification. For example, the method of FIG. 1 includes analysis of a single channel activity signal, which represents electrical activity of the ion channel, to gain an understanding of specific ion channel isoforms. This is as opposed to conventional genomic, transcriptomic, and proteomic analyses, which characterize the biological characteristics of the ion channel.

At step 110, the method includes receiving a single channel activity signal associated with an ion channel of a cell. Optionally, the cell is an electrically-excitable cell. As described in the Examples below, the single channel activity signal can be measured using a cell-attached patch-clamp system, an ion conductance microscopy-guided smart patch-clamp system, or other known technology. The cells in the Examples below are Chinese Hamster Ovary (CHO) cells. It should be understood that CHO cells are only provided as a non-limiting example of electrically-excitable cells.

At step 120, the method includes performing a time-domain analysis on the single channel activity signal. The time-domain analysis is used to extract one or more time-domain features that act as a signature for an ion channel's isoform. As described in the Examples below, the time-domain analysis can include dynamic time warping (DTW). Optionally, the time-domain analysis includes calculating a respective Euclidean distance for one or more fluctuations of an amino-acid sequence using DTW. It should be understood that the Euclidean distance for one or more fluctuations of an amino-acid sequence calculated using DTW is provided only as an example time-domain feature that provides a signature that acts as a signature for an ion channel's isoform. This disclosure contemplates using other time-domain features as a signature used to identify an ion channel's isoform. For example, additional time-domain features of the single channel activity signal may include, but are not limited to, statistical measures (e.g. mean, median, mode), waveform characteristics (e.g. shape, number of peaks and/or valleys, etc.), cross-correlation, autocorrelation, entropy, and skewness and kurtosis.

At step 130, the method includes identifying, based on the time-domain analysis, an isoform of the ion channel. As described herein, the time-domain analysis is used to extract one or more time-domain features that act as a signature for an ion channel's isoform. A non-limiting example time-domain feature is Euclidean distance for one or more fluctuations of an amino-acid sequence, which can be calculated using DTW as described above. Such one or more time-domain features facilitate the ability to distinguish between ion channels. In the Examples described herein, the ion channels are voltage-gated ion channels. For example, the identified ion channel may be one of a sodium channel (Nav), a potassium channel (Kv), a calcium channel (Cav), or a chloride channel (ClC). Alternatively or additionally, the identified ion channel may be one of a plurality of sodium ion channel forms, e.g., Nav1.5, Nav1.6, etc. Alternatively or additionally, the identified ion channel may be one of a plurality of potassium ion channel forms. Alternatively or additionally, the identified ion channel may be one of a plurality of calcium ion channels. Alternatively or additionally, the identified ion channel may be one of a plurality of chloride ion channel forms. It should be understood that sodium channels, potassium channels, calcium channels, and chloride channels are provided only as example ion channels. This disclosure contemplates using the method described with respect to FIG. 1 with other ion channels, which include, but are not limited to, voltage-gated ion channels, ligand-gated ion channels, acetylcholine-gated ion channels, and HCNs.

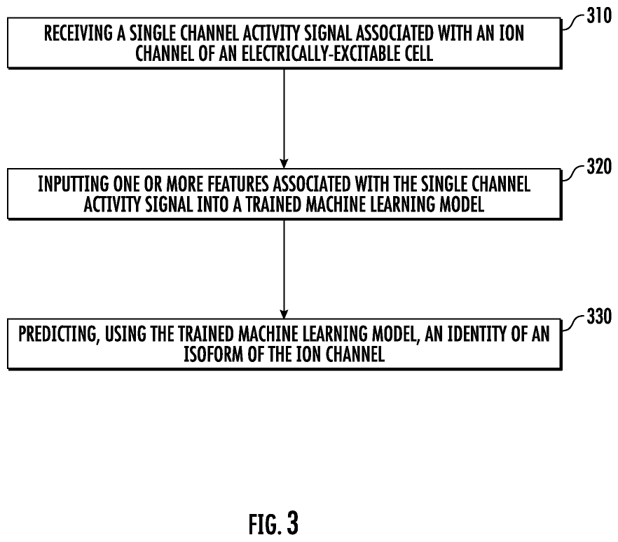

Optionally, in some implementations, the time-domain analysis can be coupled with a machine learning approach. For example, as described above, at least one time-domain feature is extracted from the single channel activity signal. A non-limiting example time-domain feature is Euclidean distance for one or more fluctuations of an amino-acid sequence. In some implementations, the method can include inputting, into a trained machine learning model, the at least one time-domain feature. The method can also include predicting, using the trained machine learning model, the isoform of the ion channel. Thus, the trained machine learning model can predict the isoform of the ion channel (i.e. target) based on the at least one time-domain feature. Alternatively, in other implementations, the method can include inputting, into a trained machine learning model, (i) one or more features associated with the single channel activity signal (e.g. raw data features) and (ii) the at least one time-domain feature. The method can also include predicting, using the trained machine learning model, the isoform of the ion channel. Thus, the trained machine learning model can predict the isoform of the ion channel (i.e. target) based on features (i) and (ii). Machine learning models can be trained as described below with regard to FIG. 3. Optionally, the method further includes comparing the isoform of the ion channel identified based on the time-domain analysis described with regard to FIG. 1 to the isoform of the ion channel predicted by the trained machine learning model. It should be understood that the time-domain analysis and machine learning prediction may confirm one another in some cases, while in other cases the time-domain analysis and machine learning prediction may be contradictory. As described herein, the trained machine learning model can be a supervised learning model such as a decision tree classifier, a support vector machine (SVM), a k-nearest neighbors (KNN) classifier, a Naïve Bayes' classifier, or an artificial neural network. Machine learning models excel in general predictions. Accordingly, the methods and systems described herein include applying machine learning prediction to precisely forecast the proportion of ion channels within the specific region under investigation. For example, trained machine learning model can accurately predict a composition such as 70% of the X isoform and 30% of the Y isoform in the designated location.

Additionally, DTW coupled with machine learning methodologies presents a robust approach for ion channel isoform identification, particularly in scenarios involving variations in the time axis. DTW facilitates the temporal alignment of ion channel activity signals, and the resulting time-domain features, such as Euclidean distances for amino acid sequence fluctuations, become critical signatures for isoform discrimination. The integration of machine learning further advances the utility of DTW-derived features. Supervised learning models excel in recognizing temporal patterns, learning to associate specific DTW features with distinct ion channel isoforms. This approach allows for the creation of predictive models capable of identifying isoforms with high accuracy. The combination of DTW and machine learning, therefore, contributes significantly to the nuanced understanding and efficient categorization of ion channel behavior, making it a valuable tool in ion channel research and biomedical applications.

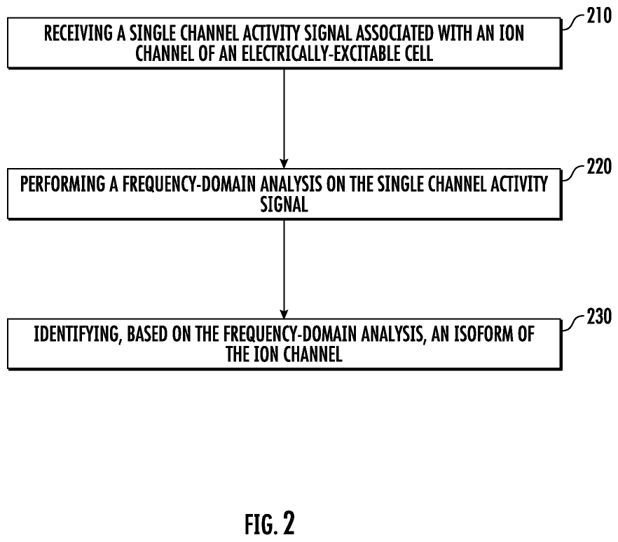

Referring now to FIG. 2, a flow chart illustrating example operations for a frequency-domain-based method for identifying ion channel isoforms is shown. This disclosure contemplates that the operations shown in FIG. 1 can be performed using a computing device (e.g., computing device 400 of FIG. 4). The frequency-domain-based signal processing approach of FIG. 2 provides improvements over conventional techniques for identifying ion channel isoforms. Such conventional techniques include genetic analysis (e.g. DNA sequencing), transcript analysis (e.g. RNA sequencing), protein analysis (e.g. Western Blotting, immunohistochemistry), and pharmacological means as described above. In contrast to conventional approaches, frequency-domain-based signal processing provides a solution that addresses the complexities of ion channel isoform identification. For example, the method of FIG. 2 includes analysis of a single channel activity signal, which represents electrical activity of the ion channel, to gain an understanding of specific ion channel isoforms. This is as opposed to conventional genomic, transcriptomic, and proteomic analyses, which characterize the biological characteristics of the ion channel.

At step 210, the method includes receiving a single channel activity signal associated with an ion channel of a cell. Optionally, the cell is an electrically-excitable cell. As described in the Examples below, the single channel activity signal can be measured using a cell-attached patch-clamp system, an ion conductance microscopy-guided smart patch-clamp system, or other known technology. The cells in the Examples below are Chinese Hamster Ovary (CHO) cells. It should be understood that CHO cells are only provided as a non-limiting example of electrically-excitable cells.

At step 220, the method includes performing a frequency-domain analysis on the single channel activity signal. Similarly as above, the frequency-domain analysis is used to extract one or more frequency-domain features that act as a signature for an ion channel's isoform. As described in the Examples below, the frequency-domain analysis includes fast Fourier transform (FFT). Alternatively, the frequency-domain analysis includes discrete Fourier transform (DFT). Optionally, the frequency-domain analysis includes determining a power spectrum of the single channel activity signal using FFT or DFT. It should be understood that power spectrum is provided only as an example frequency-domain feature that provides a signature that acts as a signature for an ion channel's isoform. This disclosure contemplates using other frequency-domain features as a signature that acts as a signature used to identify an ion channel's isoform. For example, additional frequency-domain features of the single channel activity signal may include, but are not limited to, frequency bands, total power, relative power, peak frequency, spectral centroid, spectral spread, entropy of the power spectrum, power spectral density, and skewness and kurtosis.

At step 230, the method includes identifying, based on the frequency-domain analysis, an isoform of the ion channel. As described herein, the frequency-domain analysis is used to extract one or more frequency-domain features that act as a signature for an ion channel's isoform. A non-limiting example frequency-domain feature is power spectrum as described above. Such one or more frequency-domain features facilitate the ability to distinguish between ion channels. For example, the identified ion channel may be one of a sodium channel (Nav), a potassium channel (Kv), a calcium channel (Cav), or a chloride channel (ClC). Alternatively or additionally, the identified ion channel may be one of a plurality of sodium ion channel forms, e.g., Nav1.5, Nav1.6, etc. Alternatively or additionally, the identified ion channel may be one of a plurality of potassium ion channel forms. Alternatively or additionally, the identified ion channel may be one of a plurality of calcium ion channel forms. Alternatively or additionally, the identified ion channel may be one of a plurality of chloride ion channel forms. It should be understood that sodium channels, potassium channels, calcium channels, and chloride channels are provided only as example ion channels. This disclosure contemplates using the method described with respect to FIG. 2 with other ion channels, which include, but are not limited to, voltage-gated ion channels, ligand-gated ion channels, acetylcholine-gated ion channels, and HCNs.

Optionally, in some implementations, the frequency-domain analysis can be coupled with a machine learning approach. For example, as described above, at least one frequency-domain feature is extracted from the single channel activity signal. A non-limiting example frequency-domain feature is the power spectrum. In some implementations, the method can include inputting, into a trained machine learning model, the at least one frequency-domain feature. The method can also include predicting, using the trained machine learning model, the isoform of the ion channel. Thus, the trained machine learning model can predict the isoform of the ion channel (i.e. target) based on the at least one frequency-domain feature. Alternatively, the method can include inputting, into a trained machine learning model, (i) one or more features associated with the single channel activity signal (e.g. raw data features) and (ii) the at least one frequency-domain feature. The method can also include predicting, using the trained machine learning model, the isoform of the ion channel. Thus, the trained machine learning model can predict the isoform of the ion channel (i.e. target) based on features (i) and (ii). Machine learning models can be trained as described below with regard to FIG. 3. Optionally, the method further includes comparing the isoform of the ion channel identified based on the frequency-domain analysis described with regard to FIG. 2 to the isoform of the ion channel predicted by the trained machine learning model. It should be understood that the frequency-domain analysis and machine learning prediction may confirm one another in some cases, while in other cases the frequency-domain analysis and machine learning prediction may be contradictory. As described herein, the trained machine learning model can be a supervised learning model such as a decision tree classifier, a support vector machine (SVM), a k-nearest neighbors (KNN) classifier, a Naïve Bayes' classifier, or an artificial neural network. As described above, machine learning models excel in general predictions. Accordingly, the methods and systems described herein include applying machine learning prediction to precisely forecast the proportion of ion channels within the specific region under investigation. For example, trained machine learning model can accurately predict a composition such as 70% of the X isoform and 30% of the Y isoform in the designated location.

The integration of FFT with machine learning techniques represents a powerful synergy in the context of ion channel isoform identification. FFT, by transforming time-domain signals into frequency-domain representations, extracts essential features that serve as discriminative markers for different ion channel isoforms. The frequency-domain signatures obtained through FFT become pivotal inputs for machine learning algorithms. Supervised learning models, including support vector machines and artificial neural networks, are particularly effective in recognizing patterns within the frequency data. These models learn to map the extracted features to specific ion channel isoforms during training, enabling accurate predictions and classifications. This amalgamation of FFT and machine learning not only enhances the precision of identification but also provides a versatile and scalable framework adaptable to diverse ion channel types.

Referring now to FIG. 3, a flow chart illustrating example operations for a machine learning-based method for identifying ion channel isoforms is shown. This disclosure contemplates that the operations shown in FIG. 1 can be performed using a computing device (e.g., computing device 400 of FIG. 4). The machine learning-based approach of FIG. 3 provides improvements over conventional techniques for identifying ion channel isoforms. Such conventional techniques include genetic analysis (e.g. DNA sequencing), transcript analysis (e.g. RNA sequencing), protein analysis (e.g. Western Blotting, immunohistochemistry), and pharmacological means as described above. In contrast to conventional approaches, machine learning provides a solution that addresses the complexities of ion channel isoform identification. For example, the method of FIG. 3 includes inputting features (e.g. raw data, time domain, frequency domain) of a single channel activity signal, which represents electrical activity of the ion channel, into a trained machine learning model to gain an understanding of specific ion channel isoforms. This is as opposed to conventional genomic, transcriptomic, and proteomic analyses, which characterize the biological characteristics of the ion channel.

At step 310, the method includes receiving a single channel activity signal associated with an ion channel of a cell. Optionally, the cell is an electrically-excitable cell. As described in the Examples below, the single channel activity signal can be measured using a cell-attached patch-clamp system, an ion conductance microscopy-guided smart patch-clamp system, or other known technology. The cells in the Examples below are Chinese Hamster Ovary (CHO) cells. It should be understood that CHO cells are only provided as a non-limiting example of electrically-excitable cells.

At step 320, the method includes inputting one or more features associated with the single channel activity signal into a trained machine learning model. In FIG. 3, the machine learning model is operating in inference mode. In other words, the machine learning model has already been trained with a data set (or “dataset”). This disclosure contemplates that the machine learning model is a supervised learning model. According to supervised learning, the machine learning model “learns” a function that maps an input (sometimes referred to herein as the “features”) to an output (sometimes referred to herein as the “target”) based on a data set.

As described herein, the one or more features associated with the single channel activity signal may be time-domain features or frequency-domain features. Training a machine learning model to predict ion channel isoforms (i.e. the “target”) based on time-domain features (i.e. the “features”) can be accomplished as follows. The process begins by collecting individual current traces left by the opening and closing of single ion channels in the time domain. These traces are then combined using Dynamic Time Warping (DTW) to achieve optimal matches. Subsequently, Euclidean distances are computed from these optimal matches. Similarity scores between single channel activities are calculated using Euclidian distances and translated into a histogram graph. This entire procedure is repeated for each ion channel isoform, leading to the creation of a detailed database (e.g., the training dataset). These databases (CHO1.5 or CHO1.6 isoform of Na channels) become the foundation for training machine learning algorithms. The feature set utilized in the training process includes similarity scores derived from Euclidean distances obtained from optimal matches. These features are crucial in allowing the model to discern specific characteristics associated with different ion channel isoforms. As a result, when the model encounters unknown samples, it utilizes the learned features to make accurate predictions.

An equation to compute the similarity score between two events from ion channel isoforms using the Euclidean Distance is shown below:

Similarity Score=1/(1+Euclidean Distance(n,m))

By taking the reciprocal of one plus the Euclidean distance, the formula ensures that higher similarity scores are assigned to events that are closer together (i.e., have a smaller Euclidean distance), while still maintaining a score between 0 and 1. This allows for a measure of similarity where a score closer to 1 indicates greater similarity between events, while a score closer to 0 indicates greater dissimilarity.

Additionally, training a machine learning model to predict ion channel isoforms (i.e. the “target”) based on frequency-domain features (i.e. the “features”) can be accomplished as follows. The process begins by collecting individual current traces left by the opening and closing of single ion channels in the time domain. These traces are then converted into the frequency domain (e.g. using FFT or DFT). An analysis is then performed to determine the power spectrum. This entire procedure is repeated for each ion channel isoform, leading to the creation of a detailed database (e.g., the training dataset). These databases (CHO1.5 or CHO1.6 isoform of Na channels) become the foundation for training machine learning algorithms. The power spectra serve as features during the training process. These features are crucial in allowing the model to discern specific characteristics associated with different ion channel isoforms. As a result, when the model encounters unknown samples, it utilizes the learned features to make accurate predictions.

Accordingly, the machine learning models can then trained using the datasets described above, enabling one to conduct predictions based on the time domain, the frequency domain, or both the time domain and frequency domain. The dual approach allows one to validate the results by making two independent predictions, one in the time domain and the other in the frequency domain. By integrating information from both domains, the robustness and reliability of the obtained results is enhanced.

The supervised machine learning model may be a decision tree classifier, a support vector machine (SVM), a k-nearest neighbors (KNN) classifier, a Naïve Bayes' classifier, or an artificial neural network (ANN). It should be understood that decision tree classifiers, SVMs, KNN classifiers, Naïve Bayes' classifiers, ANNs are provided only as examples. This disclosure contemplates that the machine learning model can be other supervised learning models.

A decision tree is a supervised learning model that uses a hierarchal tree structure including a root node, branches, internal nodes, and leaf nodes to predict a target. This disclosure contemplates that the decision tree can be implemented using a computing device (e.g., a processing unit and memory as described herein). Decision trees can be used for classification and regression tasks. Decision trees are trained with a data set (also referred to herein as a “dataset”) to maximize or minimize an objective function, for example a measure of the DT model's performance, during training. Decision trees are known in the art and are therefore not described in further detail herein.

A SVM is a supervised learning model that uses statistical learning frameworks to predict the probability of a target. This disclosure contemplates that the SVMs can be implemented using a computing device (e.g., a processing unit and memory as described herein). SVMs can be used for classification and regression tasks. SVMs are trained with a data set (also referred to herein as a “dataset”) to maximize or minimize an objective function, for example a measure of the SVM's performance, during training. SVMs are known in the art and are therefore not described in further detail herein.

A KNN classifier is a supervised classification model that classifies new data points based on similarity measures (e.g., distance functions). KNN classifier is a non-parametric algorithm, i.e., it does not make strong assumptions about the function mapping input to output and therefore has flexibility to find a function that best fits the data. KNN classifiers are trained with a data set (also referred to herein as a “dataset”) by learning associations between all samples and classification labels in the training dataset. KNN classifiers are known in the art and are therefore not described in further detail herein.

A Naïve Bayes' classifier is a supervised classification model that is based on Bayes' Theorem, which assumes independence among features (i.e., presence of one feature in a class is unrelated to presence of any other features). Naïve Bayes' classifiers are trained with a data set by computing the conditional probability distribution of each feature given label and applying Bayes' Theorem to compute conditional probability distribution of a label given an observation. Naïve Bayes' classifiers are known in the art and are therefore not described in further detail herein.

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation.

In some implementations, the one or more features input into the trained machine learning model include at least one time-domain feature. Time-domain features can be extracted from on the single channel activity signal by performing a time-domain analysis such as DTW. A non-limiting example time-domain feature is Euclidean distance for one or more fluctuations of an amino-acid sequence, which can be calculated using DTW as described above. Additional time-domain features of the single channel activity signal may include, but are not limited to, statistical measures (e.g. mean, median, mode), waveform characteristics (e.g. shape, number of peaks and/or valleys, etc.), cross-correlation, autocorrelation, entropy, and skewness and kurtosis. Alternatively or additionally, in some implementations, the one or more features input into the trained machine learning model include at least one frequency-domain feature. Frequency-domain features can be extracted from on the single channel activity signal by performing a frequency-domain analysis such as FFT or DFT. A non-limiting example frequency-domain feature is power spectrum as described above. Additional frequency-domain features of the single channel activity signal may include, but are not limited to, frequency bands, total power, relative power, peak frequency, spectral centroid, spectral spread, entropy of the power spectrum, power spectral density, and skewness and kurtosis. Alternatively or additionally, in some implementations, the one or more features input into the trained machine learning model include raw data. In some implementations, the features (i.e. the input to the trained machine learning model) include one or more time-domain features. In other implementations, the features (i.e. the input to the trained machine learning model) include one or more frequency-domain features. In yet other implementations, the features (i.e. the input to the trained machine learning model) include one or more raw data features. In yet other implementations, the features (i.e. the input to the trained machine learning model) include a combination of time-domain features, frequency-domain features, and/or raw data features.

At step 330, the method includes predicting, using the trained machine learning model, an identity of an isoform of the ion channel. As described herein, the machine learning model has been trained to “learn” a function that maps an input (e.g., one or more time-domain, frequency-domain, and/or raw data features) to an output (e.g., an ion channel's isoform). Thus, the one or more features input into the trained machine learning model act as a signature for an ion channel's isoform. Such features facilitate the ability to distinguish between ion channels. For example, the identified ion channel may be one of a sodium channel (Nav), a potassium channel (Kv), a calcium channel (Cav), or a chloride channel (ClC). Alternatively or additionally, the identified ion channel may be one of a plurality of sodium ion channel forms, e.g., Nav1.5, Nav1.6, etc. Alternatively or additionally, the identified ion channel may be one of a plurality of potassium ion channel forms. Alternatively or additionally, the identified ion channel may be one of a plurality of calcium ion channel forms. Alternatively or additionally, the identified ion channel may be one of a plurality of chloride ion channel forms. It should be understood that sodium channels, potassium channels, calcium channels, and chloride channels are provided only as example ion channels. This disclosure contemplates using the method described with respect to FIG. 3 with other ion channels, which include, but are not limited to, voltage-gated ion channels, ligand-gated ion channels, acetylcholine-gated ion channels, and HCNs.

Information acquired by integration the time-domain, frequency-domain, and/or machine learning approaches can be used as follows. Overall, having predictions from both time and frequency domains and machine learning models provides a more comprehensive understanding of the data and enhances the robustness of our analyses and decisions.

Comparison and Validation: Predictions from different approaches can be compared to validate the results. If the predictions align closely, it adds confidence to the findings. Any discrepancies between the predictions might indicate areas for further investigation or refinement of the analysis methods.

Integration of Results: By combining the predictions from all domains, the overall accuracy of the findings can be improved. This integration can be done in various ways, such as taking an average of the predictions or giving more weight to one prediction over the other based on their respective accuracies or reliability.

Decision Making: The predictions can inform decision-making processes depending on the specific application. For example, in medical diagnostics, if both the integrated time-domain and machine learning prediction and machine learning (raw) predictions suggest the presence of a particular condition, it may prompt further diagnostic tests or treatments.

Feedback Loop: The predictions can also serve as feedback for refining and improving the analysis methods. For example, one approach consistently produces more accurate predictions than the other. In that case, it may indicate areas where the less accurate method can be improved or where additional data or features are needed.

It should be appreciated that the logical operations described herein with respect to the various figures may be implemented (1) as a sequence of computer implemented acts or program modules (i.e., software) running on a computing device (e.g., the computing device described FIG. 4), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 4, an example computing device 400 upon which the methods described herein may be implemented is illustrated. It should be understood that the example computing device 400 is only one example of a suitable computing environment upon which the methods described herein may be implemented. Optionally, the computing device 400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 400 typically includes at least one processing unit 406 and system memory 404. Depending on the exact configuration and type of computing device, system memory 404 may be volatile (such as random access memory (RAM)), non-volatile (such as read-only memory (ROM), flash memory, etc.), or some combination of the two. This most basic configuration is illustrated in FIG. 4 by box 402. The processing unit 406 may be a standard programmable processor that performs arithmetic and logic operations necessary for operation of the computing device 400. The computing device 400 may also include a bus or other communication mechanism for communicating information among various components of the computing device 400.

Computing device 400 may have additional features/functionality. For example, computing device 400 may include additional storage such as removable storage 408 and non-removable storage 410 including, but not limited to, magnetic or optical disks or tapes. Computing device 400 may also contain network connection(s) 416 that allow the device to communicate with other devices. Computing device 400 may also have input device(s) 414 such as a keyboard, mouse, touch screen, etc. Output device(s) 412 such as a display, speakers, printer, etc. may also be included. The additional devices may be connected to the bus in order to facilitate communication of data among the components of the computing device 400. All these devices are well known in the art and need not be discussed at length here.

The processing unit 406 may be configured to execute program code encoded in tangible, computer-readable media. Tangible, computer-readable media refers to any media that is capable of providing data that causes the computing device 400 (i.e., a machine) to operate in a particular fashion. Various computer-readable media may be utilized to provide instructions to the processing unit 406 for execution. Example tangible, computer-readable media may include, but is not limited to, volatile media, non-volatile media, removable media and non-removable media implemented in any method or technology for storage of information such as computer readable instructions, data structures, program modules or other data. System memory 404, removable storage 408, and non-removable storage 410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory 404. For example, the bus may carry data to the system memory 404, from which the processing unit 406 receives and executes instructions. The data received by the system memory 404 may optionally be stored on the removable storage 408 or the non-removable storage 410 before or after execution by the processing unit 406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Signal processing methods such as machine learning, data mining, and similarity searching algorithms provide unprecedentedly rich information that can extract underlying patterns and build a predictive model of large and complex data. It has become one of the most important methods in many research areas, including medical and industrial drug research not only resolves and scales more complex biological features but also drastically reduces the resources and costs required. In the Examples below, the strength and abilities of the systems and methods described herein in ion channel research to support wet lab experiments and to get a better understanding of ion channel function and differences, which has possible fast, efficient, and easily applicable to built-in software analysis in most biomedical/electrophysiological/screening compound tools available in the market, are shown.

According to ion concentration of extracellular and intracellular regions, ion channel opening/closing time or even ion speed inside the channel (due to the electric field) may vary in time, nevertheless, all ions have to pass through the same type amino acid sequences. When ions translocate through the ion channel protein, its amino acids block the flow of ions, causing a fluctuation in the open-state ion current. The systems and methods described herein offer a means of analyzing these fluctuations to capture biological insights both in time and frequency domains. To assess how single ion channels amino acid sequences differ from one another, independent similarity searching approaches, complementary to each other, based on Dynamic Time Warping (DTW), Fast Fourier Transform (FFT), and Supervised Machine Learning are proposed. Even if the ion current fluctuations are displaced over time for any reason, the methods described herein can extract the important features of ion channels. Strikingly, a preliminary examination of these fluctuations observed within each opening exposed signatures of the primary structure of individual amino-acid sequences, allowing the differentiation of individual ion channel proteins. Data analysis enabling event idealization was performed using an offline custom-made MATLAB code based on the cumulative sums algorithm. The raw signal measurements from the sodium channel protein are pre-processed as follows: the discretization of the channel signal, splitting into separate openings and creating datasets. The strategy was put to the test to validate using a variety of datasets, such as Nav1.5, Nav1.6, and different fractional mixtures of both. The Chinese Hamster Ovary (CHO) cells were used for the electrophysiological recordings of Nav1.5 and Nav1.6 channels. The test findings demonstrate the adequacy and efficiency of the strategy for the identification and fraction prediction of sodium channel isoforms. Disclosed herein is a method that can improve the identification and screening of the most essential protein families for industrial pharmaceutical drug research and can also pave the way to clinical device fabrication in the near future.

### Methods

Both traditional cell-attached path-clamp and scanning ion conductance microscopy-guided smart patch-clamp were used. All experiments were performed at room temperature. The ion currents in voltage clamp configuration were recorded using both Axopatch 200B and 700B patch-clamp amplifier, digitized using Digidata 1440A (Molecular Devices, LLC, San Jose, USA), and acquired using pClamp 10.7 software. The single-channel late openings in each sweep were identified and analyzed using MATLAB (R2022a).

For traditional patch clamping, the single channel late openings were examined over a range of test potentials (typically −30 to −10 mV) from holding potentials (typically −100 to −120 mV). The single-channel currents were acquired at 20, 100 kHz. Each sweep was followed by a 5 second recovery period at −80 mV. Patch pipettes had a resistance of 1.2-1.6 MΩ after heat polishing, the pore diameter is around 0.8-1.2 μm.

For scanning, ion conductance microscopy-guided smart patch-clamp, the single channel late openings were examined over a range of test potentials (typically-30 mV) from a range of holding potentials (typically −100 to −120 mV). The single-channel currents were acquired at 200 kHz. Each sweep was followed by a 5 second recovery period at −5 mV. A high-resistance nanopipette (˜100 MOhm) was used to resolve the topographical structure of the cardiomyocyte surface. The nanopipette was moved to a cell or debris-free area on the dish to chop the nanopipette tip. The nanopipette resistance was continuously monitored and the chopping motion was stopped once the resistance of the pipette reached the desired resistance level is ˜20 MOhm. The clipped nanopipette was then returned to a defined area of interest on the surface (t-tubule of cardiomyocytes) and cell-attached patch clamp was performed in order to investigate single-channel late openings at that position.

The composition of the nanopipette solution was (in mM) 200 NaCl, 4 CsCl, 1 CaCl2, 2 MgCl2, 0.05 CdCl2, 10 HEPES, 0.2 NiCl2, 10 glucose, 0.03 niflumic acid, 0.004 strophanthidin, pH was adjusted at 7.4 with CsOH, thus blocking K+(with Cs+), Cl− (with niflumic acid), and cation (with CdCl2) channels, and Na+ pump (with strophanthidin), NCX (with NiCl2). Cardiomyocytes were bathed in a solution containing (in mM): 0.33 NaH2PO4, 5 HEPES, 1 CaCl2, 10 EGTA, and 140 KCl, pH 7.4 with KOH, thus depolarizing the membrane potential to ˜0 mV.

### Training Datasets

Two separate reference datasets having 400 Nav1.5 (first dataset) and 400 Nav1.6 events (second dataset) acquired using cell-attached patch clamp configuration were constructed. The event implies here electrophysiological recordings of every single channel late opening. The same datasets were used but with some changes to reflect real-world issues. Three separate datasets were created to challenge the approach described herein. The datasets used herein both time and frequency domain analysis are described below. We used eight different CHO cell data to show proof of concept.

First dataset (DS #1): It is a reference dataset having 400 Nav1.5 events.

Second dataset (DS #2): It is a reference dataset having 400 Nav1.6 events.

Third dataset (DS #3): It is an unbalanced dataset having 100 Nav1.5 events (25%) and 300 Nav1.6 events (75%).

Fourth dataset (DS #4): It is a balanced dataset having 200 Nav1.5 events (50%) and 200 Nav1.6 events (50%).

Fifth dataset (DS #5): It is an unbalanced dataset having 300 Nav1.5 events (75%) and 100 Nav1.6 events (25%).

### Dynamic Time Warping Based on Channel Identification

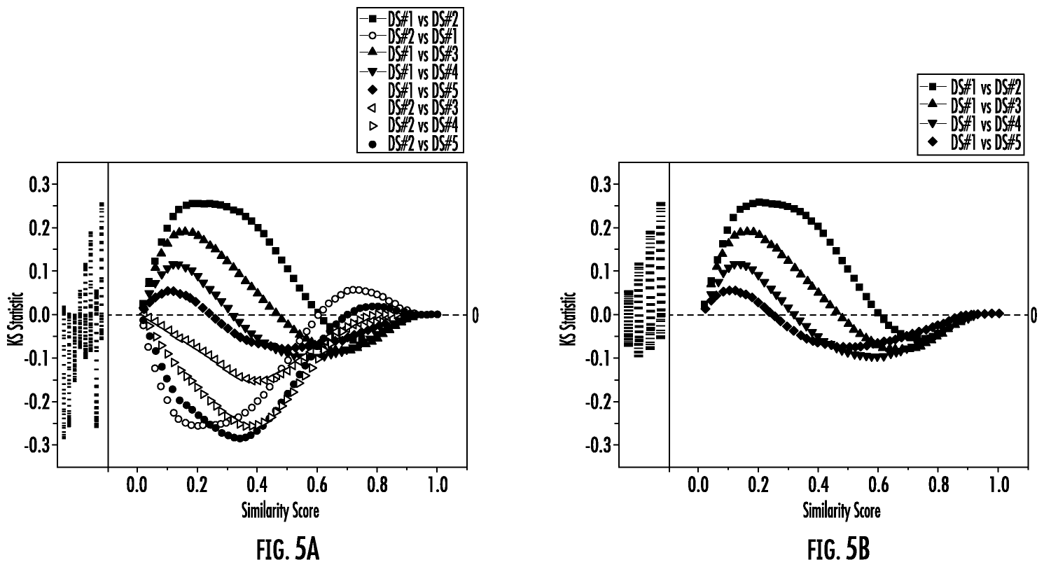

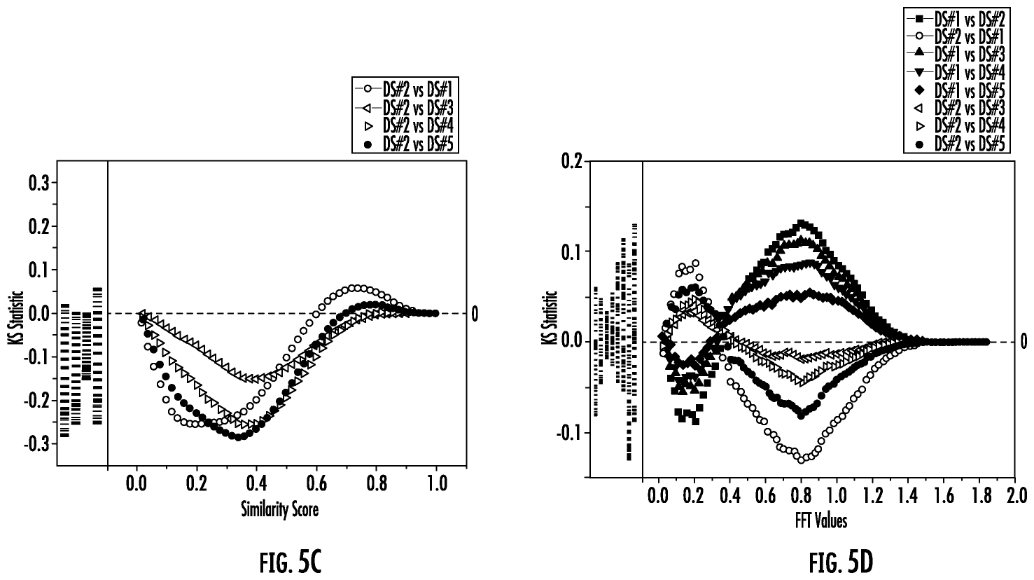

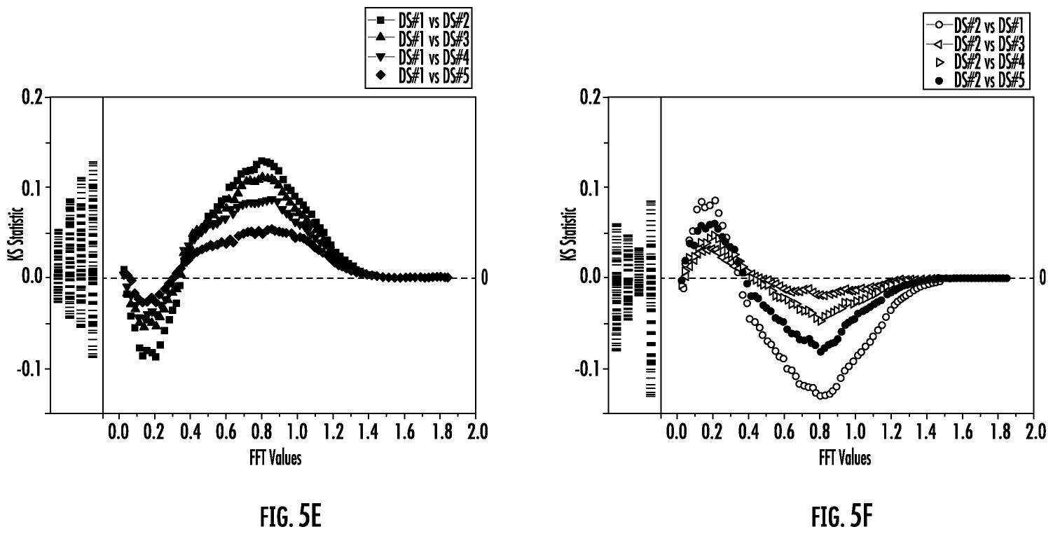

All events were scaled to the same length and the similarities between the ion channels are assessed by calculating the Euclidian distance of each fluctuation of an amino-acid sequence in the time domain using DTW. The similarity values reveal the ion channel-specific opening and closing dynamics (modes) used to identify different ion channels. The cumulative distribution function (CDF) of similarities is computed for all datasets. The Kolmogorov-Smirnov (KS) test statistic between all CDFs is calculated in the all-parameters-known case. The KS statistic between Nav1.5 and Nav1.6 and vice versa is used as a reference. Kendall correlation coefficients (KCC), use pairs of observations and determine the strength of association based on the pattern of concordance and discordance between the pairs, which are calculated between the reference and mixed datasets as fraction predictions of relevant ion channels in the mixed datasets. FIGS. 5A-5F shows the calculated KS statistic. The fraction prediction of Nav1.6 is calculated as 73% (expected 75%), 43% (expected 50%), and 21% (expected 25%), for DS #3, DS #4, and DS #5, respectively. Moreover, it is also possible to predict the total similarity fraction between mixed datasets by calculating their KCC. The total similarity fraction shows another important parameter which is the total fraction between used datasets regardless of their channel type. It is also another way to test the method. The total similarity fraction between mixed datasets is calculated as 70% (expected 75%), 48% (expected 50%), and 79% (expected 75%), for between DS #3 and DS #4, DS #3 and DS #5, DS #4 and DS #5, respectively. Table 1 (FIG. 7) shows both results of the fraction prediction of Nav1.6 in mixed datasets and the total similarity fraction between other mixed datasets.

Table 2 (FIG. 8) shows both fraction prediction of Nav1.5 in mixed datasets and the total similarity fraction between other mixed datasets. The fraction prediction of Nav1.5 is calculated as 36% (expected 25%), 52% (expected 50%), and 72% (expected 75%), for DS #3, DS #4, and DS #5, respectively. The total similarity fraction between mixed datasets is calculated as 84% (expected 75%), 64% (expected 50%), and 80% (expected 75%) between DS #3 and DS #4, DS #3 and DS #5, and DS #4 and DS #5, respectively.

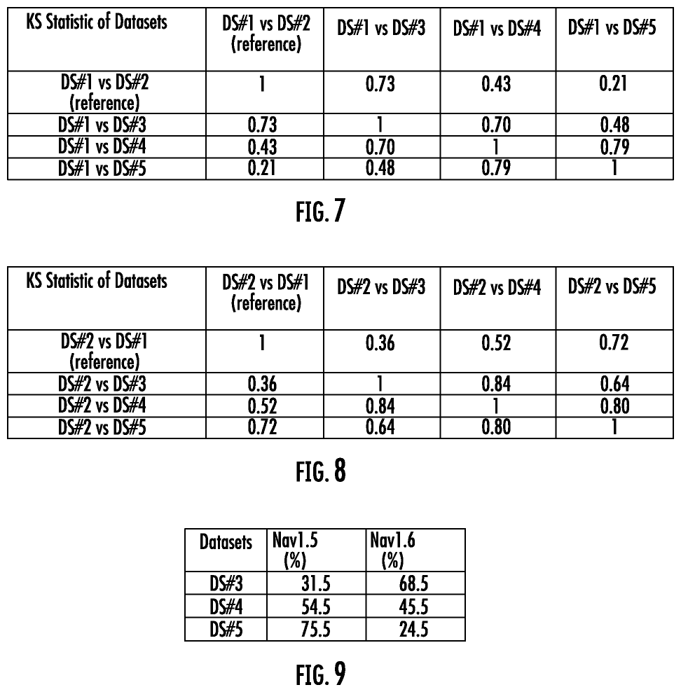

The prediction error of Nav1.6 and Nav1.5 is between 2-7% and 2-11%, respectively. More accurate predictions are made in a case with a high Nav1.5 fraction. It is possible to predict indirectly using the fraction of Nav1.5 via Nav1.6 and vice versa. The mean of both Nav1.5 and Nav1.6 fractions are calculated and the overall results are given in Table 3 (FIG. 9).

### Fast Fourier Transform Based Ion Channel Identification

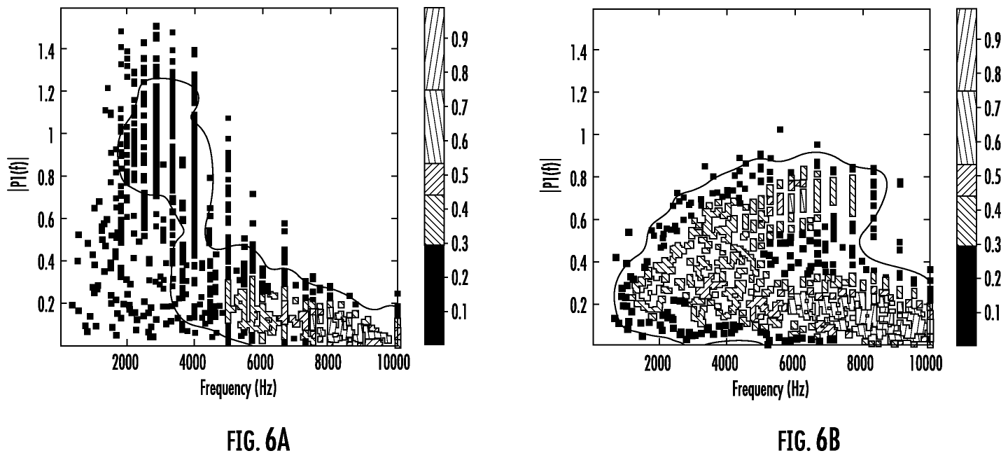

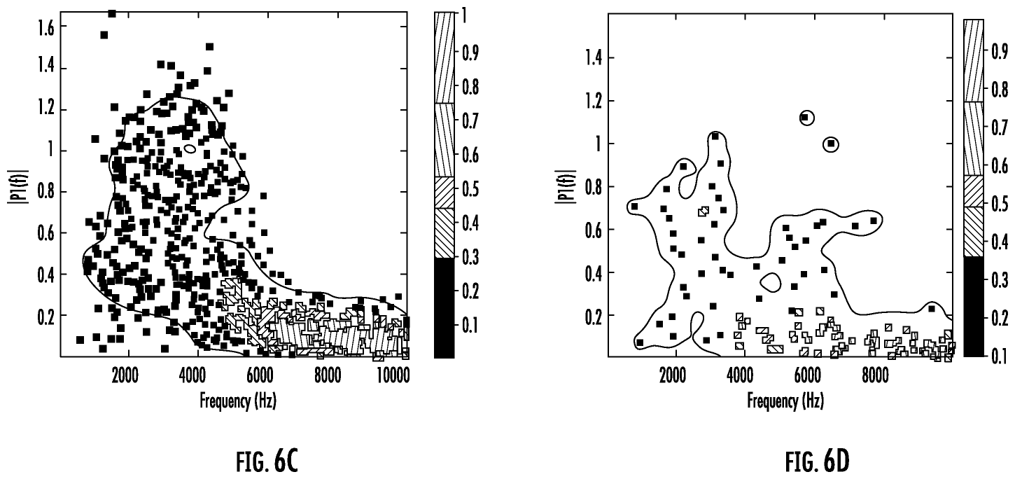

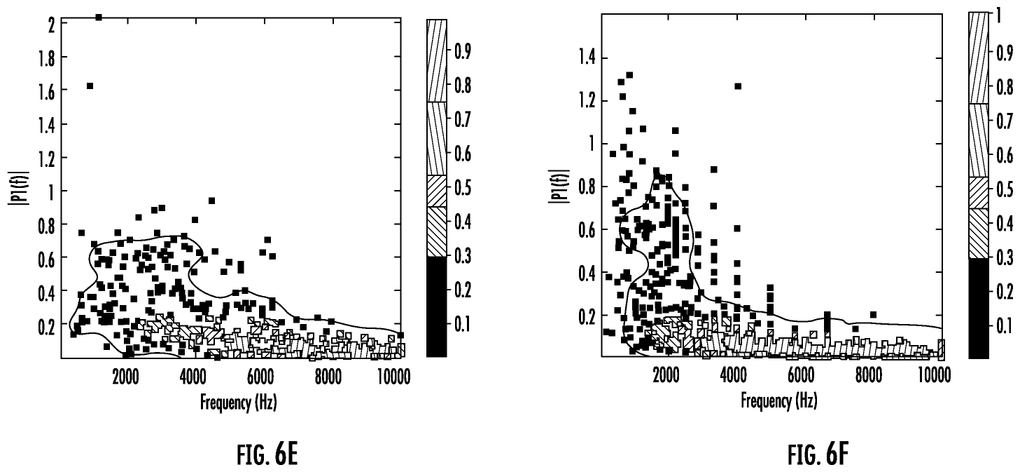

The power spectrum for all events using the Fast Fourier transform. Strikingly, Nav1.6 and Nav1.5 have unique frequency responses between 2-4 kHz and 5-8 kHz band which is used for identification, respectively. FIGS. 6A-6D show the calculated FFT results for different channels and genotypes. Not much difference in frequency responses of the two ion channels was observed above 10 kHz. These results show that when these channels are in the open state, their amino acid sequence has unique and discriminatory oscillations periodically. Nav1.6 has almost two times slower periodic oscillations than Nav1.5. On the other hand, Nav1.5 has periodic oscillations that are spread over a wider band than Nav1.6 but not as clear as Nav1.6's.

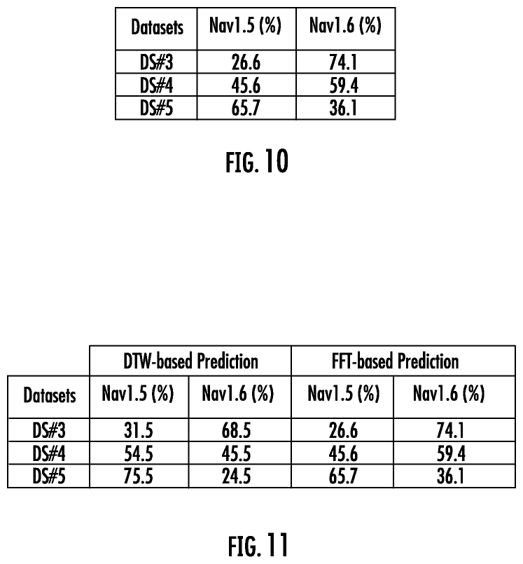

Unlike time domain analysis, the CDFs are calculated by normalizing them according to the maximum value of the FFT. Since no pairwise comparison has been used to generate CDF, the pairwise-based correlation method was not used for fraction prediction. Alternatively, the maximum and minimum of KS statistics are used as a new metric for the fraction prediction of ion channels. Similarly, the KS statistic of Nav1.5 and Nav1.6 and vice versa is used as a reference to normalize the KS statistic of mixed datasets. The mean of the normalized maximum and minimum is used to predict the fraction of related ion channels in the mixed dataset. FIGS. 5D-SF shows the calculated KS statistic. The overall results are given in Table 4 (FIG. 10).

The predicted fraction of Nav1.5 are 26.6%, 45.6%, and 65.7% for DS #3, DS #4, and DS #5, respectively. The prediction error of Nav1.5 varies between 2-9%. When the fraction of Nav1.5 increases, the prediction error increases. This can be attributed to the relatively small region of frequency responses specific to Nav1.5 in the power spectrum. The predicted fraction of Nav1.6 are 74.1%, 59.4%, and 36.1% for DS #3, DS #4, and DS #5, respectively. Since the Nav1.6 channel has a remarkable specific region in the range of 2-4 kHz band, the prediction error drops below 1% for Nav1.6 when the amount of Nav1.6 increases in the datasets. When the results of the two methods are compared, it is seen that they complement each other where they are weak. The DTW-based approach has a better prediction where mixed datasets have more Nav1.5, while in the FFT-based approach, better predictions are made in the datasets where Nav1.6 is in the higher amount. Table 5 (FIG. 11) illustrates overall results of both Nav1.5 and Nav1.6 fraction prediction for the mixed datasets using DTW and FFT-based methods.

### Machine Learning Based Ion Channel Identification

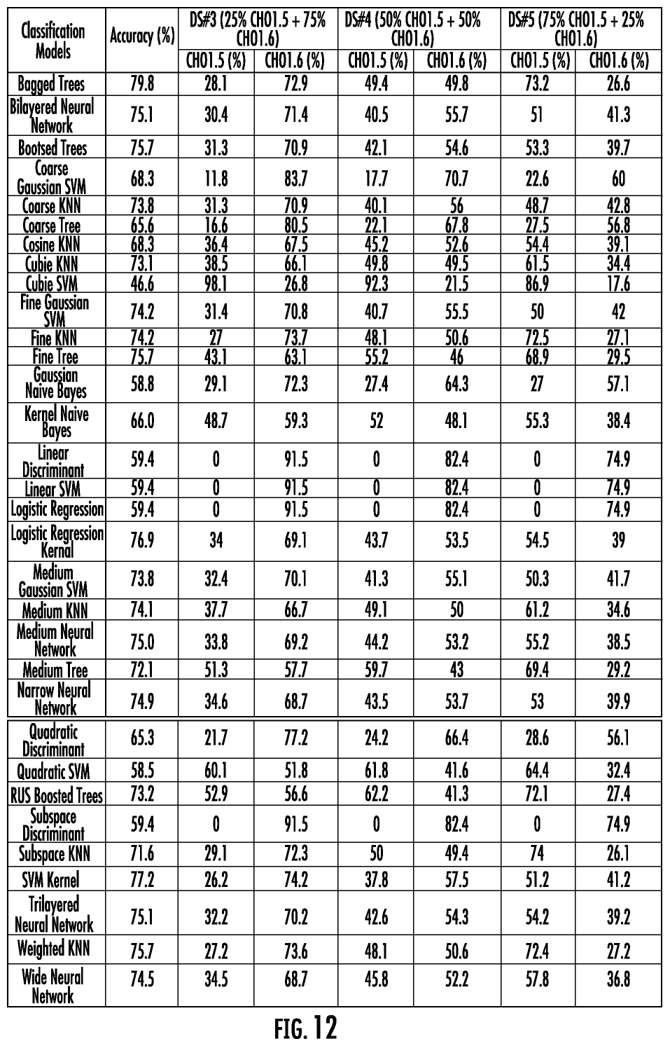

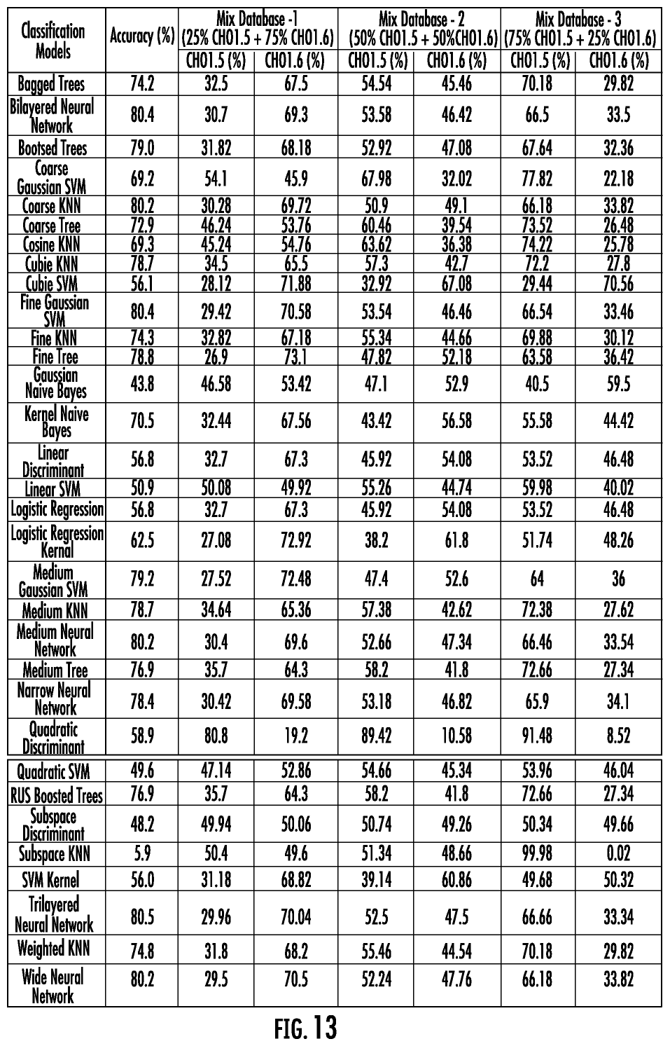

In addition to the methods proposed mentioned above, supervised machine learning has also been used to predict the fraction. Feature selection is extremely important in machine learning because they are the only measurable property of the phenomenon being observed. Choosing informative and independent features is a crucial step. Both concatenated events (raw data) and the features extracted from the FFT and DTW methods of DS #1 and DS #2 were used to train a model that generates predictions for the response to new data. Training of 32 different classification algorithms, including Bagged Trees, Bilayered Neural Network, Boosted Trees, Coarse Gaussian SVM, Coarse KNN, Coarse Tree, Cosine KNN, Cubic KNN, Cubic SVM, Fine Gaussian SVM, Fine KNN, Fine Tree, Gaussian Naive Bayes, Kernel Naïve Bayes, Linear Discriminant, Linear SVM, Logistic Regression, Logistic Regression Kernel, Medium Gaussian SVM, Medium KNN, Medium Neural Network, Medium Tree, Narrow Neural Network, Quadratic Discriminant, Quadratic SVM, RUS Boosted Trees, Subspace Discriminant, Subspace KNN, SVM Kernel, Trilayered Neural Network, Weighted KNN, Wide Neural Network were performed. Using the trained models, the CHO1.5 and CHO1.6 fractions in the DS #3, DS #4, and DS #5 datasets were predicted. Cross-validation is used to avoid overfitting. The data was partitioned into 5 disjoint sets and calculate the confusion matrix on each set. The accuracy of all models is calculated by their confusion matrix. The accuracy and prediction results of each model using the features extracted using FFT and DTW are given in Table 6 (FIG. 12) and Table 7 (FIG. 13), respectively. Bagged Trees has the highest accuracy value of 79.8%, and SVM Kernel has the second highest accuracy of 77.2% using FFT. Bagged Trees has the highest value of precision among the 5 algorithms with the highest accuracy, which is 82.0%. Next, the classifiers are evaluated in the terms of ROC curves and the area under the curve (AUC). Again Bagged Trees has the highest AUC value with 0.87. The second closest AUC value belongs to the Logistic Regression Kernel and Boosted Trees algorithms. Using the same way for DTW, the Trilayered Neural Network is the most promising classification model among all algorithms.

FIGS. 5A-5F illustrate KS test statistic between all CDFs for both FFT and DTW-based methods. CHO1.5 vs CHO1.6 and CHO1.6 vs CHO1.5 are used as a reference for CHO1.6 and CHO1.5 fraction prediction, respectively. FIG. 5A illustrates all KS statistics using DTW-based method. FIG. 5B illustrates KS statistic for CHO1.6 fraction prediction using DTW-based method. FIG. 5C illustrates KS statistic for CHO1.5 fraction prediction using DTW-based method. FIG. 5D illustrates all KS statistics using FFT-based method. FIG. 5E illustrates KS statistic for CHO1.6 fraction prediction using FFT-based method. FIG. 5F illustrates KS statistic for CHO1.5 fraction prediction using FFT-based method.

FIGS. 6A-6D illustrate FFT results of different channels and genotypes. (FIG. 6A) CHO1.6, (FIG. 6B) CHO1.5, (FIG. 6C) cardiac-specific Nav1.6 knockout, (FIG. 6D) WT.

### REFERENCES

[1] Yu, Hb., Li, M., Wang, Wp. et al. High throughput screening technologies for ion channels. Acta Pharmacol Sin 37, 34-43 (2016). https://doi.org/10.1038/aps.2015.108.
[2] Melancon K, Pliushcheuskaya P, Meiler J, Künze G. Targeting ion channels with ultra-large library screening for hit discovery. Front Mol Neurosci. 2024 Jan. 5; 16:1336004. doi: 10.3389/fnmol.2023.1336004. PMID: 38249296; PMCID: PMC10796734.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computer-implemented method comprising:
receiving a single channel activity signal associated with an ion channel of a cell;
performing a time-domain analysis on the single channel activity signal; and
identifying, based on the time-domain analysis, an isoform of the ion channel.

2. The computer-implemented method of claim 1, wherein the time-domain analysis comprises dynamic time warping (DTW).

3. The computer-implemented method of claim 2, wherein the time-domain analysis comprises calculating a respective Euclidean distance for one or more fluctuations of an amino-acid sequence using DTW.

4. The computer-implemented method of claim 1, wherein the isoform of the ion channel is one of a sodium channel (Nav), a potassium channel (Kv), a calcium channel (Cav), or a chloride channel (ClC).

5. The computer-implemented method of claim 1, wherein the single channel activity signal is measured using a cell-attached patch-clamp system or an ion conductance microscopy-guided smart patch-clamp system.

6. The computer-implemented method of claim 1, wherein performing the time-domain analysis on the single channel activity signal further comprises extracting at least one time-domain feature, the computer-implemented method further comprising:
inputting, into a trained machine learning model, the at least one time-domain feature; and
predicting, using the trained machine learning model, the isoform of the ion channel.

7. The computer-implemented method of claim 6, further comprising comparing the isoform of the ion channel identified based on the time-domain analysis to the isoform of the ion channel predicted by the trained machine learning model.

8. The computer-implemented method of claim 6, wherein the at least one time-domain feature comprises a Euclidean distance for one or more fluctuations of an amino-acid sequence.

9. The computer-implemented method of claim 6, wherein the trained machine learning model is a supervised learning model.

10. The computer-implemented method of claim 9, wherein the supervised learning model is a decision tree classifier, a support vector machine (SVM), a k-nearest neighbors (KNN) classifier, a Naïve Bayes' classifier, or an artificial neural network.

11. A computer-implemented method comprising:
receiving a single channel activity signal associated with an ion channel of a cell;
performing a frequency-domain analysis on the single channel activity signal; and
identifying, based on the frequency-domain analysis, an isoform of the ion channel.

12. The computer-implemented method of claim 11, wherein the frequency-domain analysis comprises fast Fourier transform (FFT) or discrete Fourier transform (DFT).

13. The computer-implemented method of claim 12, wherein the frequency-domain analysis comprises determining a power spectrum of the single channel activity signal using FFT or DFT.

14. The computer-implemented method of claim 11, wherein the isoform of the ion channel is one of a sodium channel (Nav), a potassium channel (Kv), a calcium channel (Cav), or a chloride channel (ClC).

15. The computer-implemented method of claim 11, wherein the single channel activity signal is measured using a cell-attached patch-clamp system or an ion conductance microscopy-guided smart patch-clamp system.

16. The computer-implemented method of claim 11, wherein performing the frequency-domain analysis on the single channel activity signal further comprises extracting at least one frequency-domain feature, the computer-implemented method further comprising:
inputting, into a trained machine learning model, the at least one frequency-domain feature; and
predicting, using the trained machine learning model, the isoform of the ion channel.

17. The computer-implemented method of claim 16, further comprising comparing the isoform of the ion channel identified based on the frequency-domain analysis to the isoform of the ion channel predicted by the trained machine learning model.

18. The computer-implemented method of claim 16, wherein the at least one frequency-domain feature comprises a power spectrum.

19. The computer-implemented method of claim 16, wherein the trained machine learning model is a supervised learning model.

20. The computer-implemented method of claim 19, wherein the supervised learning model is a decision tree classifier, a support vector machine (SVM), a k-nearest neighbors (KNN) classifier, a Naïve Bayes' classifier, or an artificial neural network.

21-33. (canceled)
